(19) United States
(12) Patent Application Publication
(10) Pub. No.: US 2022/0202937 A1
(43) Pub. Date: Jun. 30, 2022

(54) Novel Formulations Which Stabilize Low Dose Antibody Compositions

(71) Applicant: Janssen Biotech, Inc., Horsham, PA (US)

(72) Inventor: Joanna Mackay, Littlerock, WA (US)

(21) Appl. No.: 17/503,759

(22) Filed: Oct. 18, 2021

Related U.S. Application Data
(63) Continuation of application No. 16/145,324, filed on Sep. 28, 2018, now abandoned.
(60) Provisional application No. 62/565,178, filed on Sep. 29, 2017.

(51) Int. Cl. A61K 39/395; A61K 9/08; A61K 9/00; A61K 47/02; A61K 47/12; A61K 47/18; A61K 47/22; A61K 47/26; B65D 65/42; C07K 16/28

(52) U.S. Cl. CPC A61K 39/39591; A61K 9/08; A61K 9/0019; A61K 47/02; A61K 47/12; A61K 2039/54; A61K 47/22; A61K 47/26; B65D 65/42; C07K 16/2809; C07K 2317/94; A61K 47/183

(57) ABSTRACT

The present invention addresses an ongoing need in the art to improve the stability of antibody compositions. The invention broadly relates to novel formulations which stabilize and inhibit protein adsorption of low dose antibody compositions in a container means comprising a coating. More particularly, the invention described hereinafter, addresses a need in the art for formulations which stabilize and inhibit protein adsorption of low dose antibody compositions which are processed, developed, formulated, manufactured and/or stored in container means such as fermentors, bioreactors, vials, flasks, bags, syringes, rubber stoppers, tubing and the like.

### CROSS-REFERENCE TO RELATED APPLICATIONS

This application is a Continuation of U.S. application Ser. No. 16/145,324, filed 28 Sep. 2018, which claims the benefit of U.S. Provisional Application Ser. No. 62/565,178, filed 29 Sep. 2017. The entire contents of the aforementioned applications are incorporated herein by reference in their entireties.

### TECHNICAL FIELD

The present invention generally relates to the fields of immunology, oncology, antibody formulation, protein stability and process development. More particularly, the invention relates to novel formulations which inhibit protein adsorption of low dose antibody compositions to a container comprising a coating.

### BACKGROUND OF THE INVENTION

Biopharmaceuticals approved as safe and effective and those in clinical development have varying dosage schedules. Accordingly, these biopharmaceuticals are provided in drug product formulations with varying concentrations and volumes dependent on the amount of protein that must be dosed. Low concentration protein formulations can be rendered unstable if a significant portion of the drug substance protein adsorbs onto the surface of a container such as a vial or syringe. Further, the change in active protein concentration can lead to administration of a lower dose to a patient than what is expected. In addition, a number of biopharmaceuticals are formulated and provided ready for clinical administration without further manipulation; however, many products require varying degrees of handling by the nurse, pharmacist, and/or physician. During handling and administration, physical and chemical stability of a protein in the drug product formulation must be maintained. Loss of stability can occur when the protein formulation is diluted to low concentrations with intravenous (i.v.) solutions, thus lowering the excipient concentration and modifying the composition and properties of the original drug product formulation. When delivering a biopharmaceutical product via the i.v. route, several factors must be considered including protein biophysicochemical properties, formulation composition, concentration of the active protein to be delivered, choice of diluent, contact surfaces, and infusion time and rate. Here, contact surfaces are of particular interest as proteins will minimize the interfacial energy due to their amphiphilic nature. With the widespread use of a variety of plastic polymers in syringes and i.v. infusion containers and lines, the risk of protein loss by adsorption is significant, especially at low doses. Thus, protein loss due to adsorption onto filters, containers, syringes, and tubing must be investigated and addressed during drug product development, particularly for low dose products.

Thus, there is a need for formulations suitable for low dose proteins.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject.

The present invention broadly relates to novel formulations which stabilize and inhibit protein adsorption of low dose antibody compositions to a container means. In one specific embodiment, the invention is directed to novel formulations which stabilize low dose antibody compositions against container-coating interactions, shear forces, shipping agitation, and

[symbol]

In certain embodiments, the invention is directed to formulations which stabilize a composition, the formulation comprising (i) a pH buffered solution with a pH of about 5 to about 7.4, (ii) a polysorbate and (iii) one or more antibodies at a dose of less than about 70 μg, wherein the formulation is comprised in a container means with a coating. In certain embodiments, the pH buffered solution of the formulations has a pH of 5 to about 7.4. In other embodiments, the buffer is histidine, phosphate, or acetate. In another embodiment, the polysorbate of the formulation is selected from the group consisting of polysorbate 20 (Tween™ 20), and polysorbate 80 (Tween™ 80). In one particular embodiment, the surfactant is polysorbate 80. In certain embodiments, the final concentration of the polysorbate 80 in formulation is at least 0.001% to 0.1% polysorbate 80 weight/volume of the formulation. In certain embodiments, the final concentration of the polysorbate 20 in formulation is at least 0.001% to 0.1% polysorbate 20 weight/volume of the formulation.

In one specific embodiment, the antibody formulation is contained in a container means. In certain embodiments, the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable injector pen. In certain embodiments, the container means is coated. In particular embodiments, the container means has a silicon dioxide coating. In other embodiments, the container means has a hydrophobic coating. In certain embodiments, the antibody formulation further comprises a sugar. In other embodiments, the formulation further comprises a sugar alcohol. In another embodiment, the antibody formulation further comprises L-methionine or ethylenediaminetetraacetic acid (EDTA).

In another embodiment, the invention is directed to formulations which stabilize a an anti-CD3 bispecific antibody composition, the formulation comprising (i) a pH buffered solution with a pH of about 5 to about 7.4, (ii) a polysorbate and (iii) an anti-CD3 bispecific antibody at a dose of less than about 70 μg, wherein the formulation is contained in a container means with a coating. In certain embodiments, the pH buffered solution of the formulations has a pH of 5 to 7.4. In other embodiments, the buffer is histidine, phosphate, or acetate. In another embodiment, the polysorbate of the formulation is selected from the group consisting of polysorbate 20 (Tween™ 20), and polysorbate 80 (Tween™ 80). In one particular embodiment, the surfactant is polysorbate 80. In certain embodiments, the final concentration of the polysorbate 80 in formulation is at least 0.001% to 0.1% polysorbate 80 weight/volume of the formulation. In certain embodiments, the final concentration of the polysorbate 20 in formulation is at least 0.001% to 0.1% polysorbate 20 weight/volume of the formulation.

In one embodiment, the anti-CD3 bispecific antibody formulation is contained in a container means. In some embodiments, the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable pen. In certain embodiments, the container means is coated. In particular embodiments, the container means has a hydrophobic coating. In other embodiments, the antibody formulation further comprises a sugar. In another embodiment, the formulation further comprises a sugar alcohol. In another embodiment, the antibody formulation further comprises L-methionine or EDTA.

In another aspect, the invention provides a method for containing an antibody composition comprising providing an antibody composition ready for injection and comprising at least one antibody as an active ingredient at a dose of less than about 70 μg into a container means with a coating wherein the pharmaceutical composition is in a formulation comprising (i) a pH buffered solution with a pH of about 5 to about 7.4 and (ii) a polysorbate.

Other features and advantages of the invention will be apparent from the following detailed description, from the embodiments thereof, and from the claims.

### DETAILED DESCRIPTION OF THE INVENTION

### Definitions

As used herein, the term “about” is used to modify, for example, the quantity of an ingredient in a composition, concentration, volume, process temperature, process time, yield, flow rate, pressure, and ranges thereof, employed in describing the invention. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and other similar considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include such equivalents.

As used herein “antibody” refers to all isotypes of immunoglobulins (IgG, IgA, IgE, IgM, IgD, and IgY) including various monomeric, bispecific, polymeric and chimeric forms, unless otherwise specified. Specifically encompassed by the term “antibody” are polyclonal antibodies, monoclonal antibodies (mAbs), and antibody-like polypeptides, such as dual-affinity re-targeting (DART) molecules, single chain antibodies, human antibodies, antibody fragments, chimeric antibodies and humanized antibodies.

The term “pharmaceutical agent or drug” as used herein refers to a chemical compound or composition capable of inducing a desired therapeutic effect when properly administered to a patient.

The phrase “pharmaceutically acceptable” means approved by a regulatory agency of the Federal or a state government, or listed in the U.S. Pharmacopeia, European Pharmacopeia, or other generally recognized pharmacopeia for use in animals, in particular, for use in humans.

The terms “stability”, “stable”, and “stabilize” as used herein in the context of a liquid formulation comprising an antibody (including antibody fragment thereof) that specifically binds to an antigen of interest refer to the resistance of the antibody (including antibody fragment thereof) in the liquid antibody formulation to lose its bioactivity or physical integrity (e.g., aggregation, precipitation, adsorption), when prepared under the manufacture, preparation, transportation and storage conditions of the invention. The “stable” formulations of the invention demonstrate an improved biophysical and chemical integrity profile under given manufacture, preparation, transportation and storage conditions as compared to a reference formulation.

Unless otherwise defined, scientific and technical terms used herein shall have the meanings that are commonly understood by those of ordinary skill in the art. Further, unless otherwise required by context, singular terms shall include pluralities and plural terms shall include the singular.

### Formulations

The present invention addresses an ongoing need in the art to improve the stability of antibody compositions while in solution. Thus, the present invention broadly relates to novel formulations which stabilize protein and antibody compositions. More particularly, the invention described hereinafter, addresses a need in the art for formulations which stabilize and inhibit protein adsorption of antibody compositions which are processed, developed, formulated, manufactured and/or stored in container means such as fermentors, bioreactors, vials, flasks, bags, syringes, rubber stoppers, tubing and the like. As set forth above in the Background of the Invention, various factors influence the stability of antibody compositions, including, but not limited to, chemical stability of the antibody composition, physical/thermal stability of the antibody composition, compatibility of the antibody composition with the container/closure system, interactions between antibody composition and inactive ingredients (e.g., buffers, salts, excipients, cryoprotectants), manufacturing processes, dosage form, dosage amounts, environmental conditions encountered during shipping, storage and handling (e.g., temperature, humidity, shear forces), and the length of time between manufacture and usage.

The stability of an antibody composition of the invention is readily determined using standard techniques, which are well known and routine to those of skill in the art. For example, an antibody composition is assayed for stability, aggregation, activity, particulate formation, protein (concentration) loss, and the like, by methods including, but not limited to, light scattering, optical density, sedimentation velocity centrifugation, sedimentation equilibrium centrifugation, circular dichroism (CD), Lowry assay, bicinchoninic acid (BCA) assay, antibody binding, and the like.

As set forth in detail herein, the present invention relates to the unexpected and surprising results that formulating a low dose (<70 μg) antibody composition with a surfactant such as Tween™ 20 or Tween™ 80 significantly enhances stability and inhibits protein adsorption to a container means with a coating. For example, it was observed in the present invention (e.g., see Example 2), that the dual-affinity re-targeting molecule known as duvortuxizumab at a dose of about 30 μg, formulated in pH 5.2 acetate buffer with 9% sucrose, 0.4 mg/mL L-methionine, and 50 μM EDTA and filled in an uncoated vial, had only a maximum protein recovery of 36% after three days of gentle agitation via a horizontal orbital shaker. (The horizontal orbital shaker was used to simulate typical process, shipping and storage conditions of an antibody composition). The same formulation filled in various types of coated vials had a maximum protein recovery of 76%. However, it was surprisingly observed that duvortuxizumab at a dose of about 30 μg, formulated in pH 5.2 acetate buffer with 9% sucrose, 0.4 mg/mL L-methionine, 50 μM EDTA, and 0.01% Tween™ 80, filled in various types of coated vials had at least 97% protein recovery. Thus, these data demonstrate that the combination of surfactant (e.g., Tween™ 80) plus coated container means for a low dose antibody composition formulation enhances the stability of the antibody composition.

In these experiments, the antibody compositions were filled in various coated and non-coated container means (e.g., see Tables 3 to 6) and subjected to simulated shipping and handling conditions via agitation. It was observed in these experiments that the container means comprising a coating exhibited a higher percentage of protein recovery when compared to an uncoated container. Furthermore, it was observed that for formulations containing 70 μg protein or less, both the use of a container means comprising a coating and the addition of a surfactant was necessary to achieve protein recovery higher than 90%, particularly if the formulation pH was below the isoelectric point of the protein.

Thus, the invention as set forth herein, is directed to novel formulations which stabilize low dose antibody compositions. These low dose antibody compositions can include bispecific CD3 redirection constructs such as duvortuximab, anti-IL1RAP x CD3 (WO2017079121), anti-BCMA x CD3 (WO2017031104), anti-CD123 x CD3 (WO2016036937), anti-PSMA x CD3 (WO2016179518), and anti-ROR1 x CD3 (WO2017127499). The compositions can also include low dose formulations of any other antibody. The novel formulations disclosed herein stabilize low dose antibody compositions in a container means comprising a coating, against the various factors which influence the stability of antibody compositions (e.g., shear forces, shipping agitation, container interactions, adsorption, absorption, manufacturing processes, temperature, humidity, length of time between manufacture and usage, etc.).

In certain embodiments, the invention is directed to a formulation which stabilizes a low dose antibody composition, the formulation comprising a pH buffered solution with a pH of about 5 to about 7.4, a polysorbate, and one or more antibodies, wherein the formulation is comprised in a container means with a coating.

In another embodiment, the invention is directed to a formulation which stabilizes an anti-CD3 bispecific antibody composition, the formulation comprising (i) a pH buffered solution with a pH of about 5 to about 7.4 and (ii) a polysorbate, and (iii) an anti-CD3 bispecific antibody at a dose of less than about 70 μg, wherein the formulation is comprised in a container means with a coating.

In another embodiment, the invention is directed to a formulation which stabilizes a duvortuxizumab composition, the formulation comprising (i) a pH buffered solution with a pH of about 5 to about 7.4 and (ii) a polysorbate, and (iii) duvortuxizumab at a dose of less than about 70 μg, wherein the formulation is comprised in a container means with a coating. Duvortuxizumab is described in WO201/6048938 incorporated herein by reference.

In another embodiment, the invention is directed to a formulation which stabilizes a ichorcumab composition, the formulation comprising (i) a pH buffered solution with a pH of about 5 to about 7.4 and (ii) a polysorbate, and (iii) ichorcumab at a dose of less than about 70 μg, wherein the formulation is comprised in a container means with a coating. Ichorcumab is an anti-thrombin antibody described in U.S. Pat. No. 9,518,129 incorporated herein by reference.

In another embodiment, the invention is directed to a formulation which stabilizes a daratumumab composition, the formulation comprising (i) a pH buffered solution with a pH of about 5 to about 7.4 and (ii) a polysorbate, and (iii) daratumumab at a dose of less than about 70 μg, wherein the formulation is comprised in a container means with a coating. Daratumumab is described in U.S. Pat. No. 7,829,673 incorporated herein by reference.

In another embodiment, the invention is directed to a formulation which stabilizes a ustekinumab composition, the formulation comprising (i) a pH buffered solution with a pH of about 5 to about 7.4 and (ii) a polysorbate, and (iii) ustekinumab at a dose of less than about 70 μg, wherein the formulation is comprised in a container means with a coating. Ustekinumab is described in U.S. Pat. No. 6,902,734 incorporated herein by reference.

In another embodiment, the invention is directed to a formulation which stabilizes an sirukumab composition, the formulation comprising (i) a pH buffered solution with a pH of about 5 to about 7.4 and (ii) a polysorbate, and (iii) at a dose of less than about 70 μg, wherein the formulation is comprised in a container means with a coating. Sirukumab is described in U.S. Reissue 43,672 incorporated herein by reference.

In another embodiment, the invention is directed to a formulation which stabilizes an anti-NKG2D antibody composition, the formulation comprising (i) a pH buffered solution with a pH of about 5 to about 7.4 and (ii) a polysorbate, and (iii) an anti-NKG2D antibody at a dose of less than about 70 μg, wherein the formulation is comprised in a container means with a coating. In one embodiment, said anti-NKG2D antibody is described in U.S. Pat. No. 7,879,985 incorporated herein by reference.

In another embodiment, the invention is directed to a formulation which stabilizes an anti-IL1RAP x CD3 bispecific antibody composition, the formulation comprising (i) a pH buffered solution with a pH of about 5 to about 7.4 and (ii) a polysorbate, and (iii) an anti-IL1RAP x CD3 bispecific antibody at a dose of less than about 70 μg, wherein the formulation is comprised in a container means with a coating. In one embodiment, said IL1RAP x CD3 bispecific antibody is described in WO2017079121 incorporated herein by reference.

In another aspect, the invention provides a method for containing an antibody composition comprising providing an antibody composition ready for injection and comprising at least one antibody as an active ingredient at a dose of less than about 70 μg into a container means with a coating wherein the pharmaceutical composition is in a formulation comprising (i) a pH buffered solution with a pH of about 5 to about 7.4 and (ii) a polysorbate.

### Surfactants

As set forth above, the invention is directed to formulations which stabilize antibody compositions against the various factors which influence the stability of antibody compositions (e.g., shear forces, shipping agitation, silicone oil interactions, adsorption, manufacturing processes, temperature, humidity, length of time between manufacture and usage, etc.). In certain embodiments, the invention is directed to formulations comprising a surfactant.

A surfactant (or a surface-active agent) is generally defined as (a) an amphiphilic molecule or compound comprising a hydrophilic group or moiety and a lipophilic (hydrophobic) group or moiety and/or (b) a molecule, substance or compound that lowers or reduces surface tension of a solution. As defined herein, a “surfactant” of the present invention is any molecule or compound that lowers the surface tension of an antibody composition formulation.

A surfactant used in a formulation of the present invention comprises any surfactant or any combination of surfactants which stabilizes and inhibits protein adsorption to a container of an antibody composition described herein. Thus, a surfactant of the invention includes, but is not limited to, polysorbate 20 (Tween™ 20), polysorbate 40 (Tween™ 40), polysorbate 60 (Tween™ 60), polysorbate 65 (Tween™ 65), polysorbate 80 (Tween™ 80), polysorbate 85 (Tween™ 85), Triton™ N-101 , Triton™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (Cremophor EL™), soy lecithin, poloxamer, hexadecylamine, octadecylamine, octadecyl amino acid esters, lysolecithin, dimethyl-dioctadecylammonium bromide, methoxyhexadecylgylcerol, pluronic polyols, polyamines (e.g., pyran, dextransulfate, poly IC, carbopol), peptides (e.g., muramyl peptide and dipeptide, dimethylglycine, tuftsin), oil emulsions, mineral gels (e.g., aluminum phosphate) and immune stimulating complexes (ISCOMS).

A person of skill in the art may readily determine a suitable surfactant or surfactant combination by measuring the surface tension of a particular antibody composition formulation in the presence and absence of the surfactant(s). Alternatively, a surfactant is evaluated qualitatively (e.g., visual inspection of particulate formation) or quantitatively (e.g., light scattering, sedimentation velocity centrifugation, optical density, antigenicity) for its ability to reduce, inhibit or prevent adsorption of an antibody composition.

### Container Means

In certain embodiments, the invention is directed to formulations of antibody compositions contained in a container means. As defined herein, a “container means” of the present invention includes any composition of matter which is used to “contain”, “hold”, “mix”, “blend”, “dispense”, “inject”, “transfer”, “nebulize”, etc. an antibody composition during research, processing, development, formulation, manufacture, storage and/or administration. For example, a container means of the present invention includes, but is not limited to, general laboratory glassware, flasks, beakers, graduated cylinders, fermentors, bioreactors, tubings, pipes, bags, jars, vials, vial closures (e.g., a rubber stopper, a screw on cap), ampoules, syringes, syringe stoppers, syringe plungers, rubber closures, plastic closures, glass closures, and the like. A container means of the present invention is not limited by material of manufacture, and includes materials such as coated or uncoated glass.

The skilled artisan will appreciate that the container means set forth above are by no means an exhaustive list, but merely serve as guidance to the artisan with respect to the variety of container means which are used to contain, hold, mix, blend, dispense, inject, transfer, nebulize, etc. an antibody or antibody composition during research, processing, development, formulation, manufacture, storage and/or administration of the composition. Additional container means contemplated for use in the present invention may be found in published catalogues from laboratory equipment vendors and manufacturers such as United States Plastic Corp. (Lima, Ohio), VWR™ (West Chester, Pa.), BD Biosciences (Franklin Lakes, N.J.), Fisher Scientific International Inc. (Hampton, N.H.), Schott™ (Mainz, Germany), Omni Glass (Billerica, Mass.), West Pharma (Exton, Pa.). Gerresheimer (Dusseldorf, Germany), and Sigma-Aldrich (St. Louis, Mo.).

There is a wealth of general knowledge regarding surfaces and or coatings for container means. A non-exhaustive list includes polyethylene oxide/glycol-like and other coatings deposited via plasma assisted chemical vapor deposition—see, for example, Erika E. Johnston E. E., Bryers J. D., Ratner B. D. Langmuir 2005, 21, 870-881; Sardella E., Gristina R., Senesi G. S., d'Agostino R., Favia P. Plasma Process. Polym. 2004, 1, 63-72; Shen M., Martinson L., Wagner M. S., Castner D. G., Ratner B. D., Horbett T. A. J. Biomater. Sci. Polymer Edn. 2002, 13, 367-390; Shen M., Pan Y. V., Wagner M. S., Hauch K. D., Castner D. G., Rather B. D., Horbett T. A. J. Biomater. Sci. Polymer Edn. 2001, 12, 961-978; U.S. Pat. No. 5,153,072; Lopez G. P., Ratner B. D. J. Polym. Sci. A—Polym. Chem. 1992, 30, 2415-2425; and U.S. Pat. No. 5,002,794. For (derivatized) alkanethiol coatings deposited see, for example, Li L. Y,, Chen S. F., Rather B. D., Jiang S. Y. J. Phys. Chem. B 2005, 104, 2934-2941; Chirakul P., Pérez-Luna V. H., Owen H., López G. P. Langmuir 2002, 18, 4324-4330; Prime K. L., Whitesides G. M. J. Am. Chem. Soc. 1993, 115, 10714-10721; Pale-Grosdemange C., Simon F. S., Prime K. L., Whitesides G. M. J. Am. Chem. Soc. 1991, 113, 12-20. For organosilane coatings see, for example, Seigers C., Biesalski M., Haag R. Chem. Eur. J. 2004, 10, 2831-2838; US 2003/0092879; Yang Z., Galloway J. A., Yu H. Langmuir 1999, 15, 8405-8411; Lee S. W., Laibinis P. E. Biomaterials 1998, 19, 1660-1675; and U.S. Pat. No. 6,235,340. For hydrogel coatings see, for example, U.S. Pat. No. 6,844,028. For poly-L-lysine/polyethylene glycol coatings see, for example, US 2002/0128234; Huang N. P., Michel R., Voros J., Textor M., Hofer R., Rossi A., Elbert D. L., Hubbell J. A., Spencer N. D. Langmuir 2001, 17, 489-498; Kenausis G. L. Vörös J., Elbert D. L., Huang N., Hofer R, Ruiz-Taylor L., Textor M., Hubbell J. A., Spencer N. D. J. Phys. Chem. B 2000, 104, 3298-3309. For polyethylene oxide graft coatings see, for example, Sofia S. J., Premnath. V., Merrill E. W. Macromolecules 1998, 31, 5059-5070. These examples represent but are not an exhaustive compilation of the large number of available surface treatment and/or coating possibilities.

Currently, no commercially available pharmaceutical package (coated or uncoated) contains all of the favorable protein adsorption deterring characteristics described above, but tend to have a few desirable ones while still having some that promote, rather than deter, protein adsorption. While glass (borosilicate, soda-lime, etc.) is hydrophilic and hydrogen bond accepting, it is highly ionic and has no steric hindrance to deter protein binding. The high density of negative charges under liquid formulation conditions (pH 5-9) on the surface will promote the ionic binding of positively charged residues on the proteins (i.e. lysine, histidine, and the amino terminus). The siliconization of glass to passivate the surface and provide lubricity in syringes results in a relatively non-ionic surface that is sterically blocked, but the silicone oil renders the surface very hydrophobic while decreasing its hydrogen bond accepting ability. Silicone oil treatment can also result in the generation of unwanted particulate matter in syringes as silicone droplets that leave the surface and enter the solution. Hydrophobic surfaces tend to exclude water and facilitate the adsorption of proteins. The hydrophobicity of the environment the proteins encounter can also lead to protein denaturation as the hydrophobic core of the proteins seeks to interact with the surface and unfold its native structure to obtain a minimum free energy conformation. Hydrophobic coatings containing fluorine with anti-adherency properties for solutions/suspensions containing medicinally relevant particles/agglomerates have been prepared previously by plasma enhanced chemical vapor deposition—see, for example, U.S. Pat. No. 6,599,594.

Thus, the novel formulations of the present invention are particularly advantageous in that they stabilize and inhibit adsorption of low dose antibody formulations comprised in a container means throughout the various stages of research, processing, development, formulation, manufacture, storage and/or administration of the composition. The novel formulations of the invention not only stabilize antibody compositions against physical/thermal stresses (e.g., temperature, humidity, shear forces, etc.), they also enhance stability and inhibit adsorption of antibody compositions against negative factors or influences such as incompatibility of the antibody composition with the container/closure system (e.g., a siliconized container means). Thus, the novel formulations of the present invention are particularly useful in stabilizing the antibody composition.

### Excipients and pH

The formulations of the antibody compositions in the present invention comprise one or more excipients. The term “excipient,” as used herein, means any non-therapeutic agent added to the formulation to provide a desired consistency, viscosity or stabilizing effect.

In certain embodiments, the pharmaceutical formulation of the invention comprises a buffer suitable to maintain a pH ranging from about 5 to about 7.4. An exemplary buffer suitable for use in the formulations of the present invention include, e.g. a histidine or acetate buffer. In one embodiment, the histidine buffer is prepared at a concentration of 10 mM.

The amount of histidine contained within the formulations of the present invention may vary from about 1 mM to about 50 mM; about 2 mM to about 20 mM; about 3 mM to about 12 mM; or about 10 mM.

The pharmaceutical formulations of the present invention may also comprise one or more carbohydrates, e.g., one or more sugars. The sugar can be a reducing sugar or a non-reducing sugar. “Reducing sugars” include, e.g., sugars with a ketone or aldehyde group and contain a reactive hemiacetal group, which allows the sugar to act as a reducing agent.

Specific examples of reducing sugars include fructose, glucose, glyceraldehyde, lactose, arabinose, mannose, xylose, ribose, rhamnose, galactose and maltose. Non-reducing sugars can comprise an anomeric carbon that is an acetal and is not substantially reactive with amino acids or polypeptides to initiate a Maillard reaction. Specific examples of non-reducing sugars include sucrose, trehalose, sorbose, sucralose, sorbitol, mannitol, melezitose and raffinose. Sugar acids include, for example, saccharic acids, gluconate and other polyhydroxy sugars and salts thereof.

The amount of sugar contained within the formulations of the present invention will vary depending on the specific circumstances and intended purposes for which the formulations are used. In certain embodiments, the formulations may contain about 0.1% to about 20% sugar; about 0.5% to about 20% sugar; about 1% to about 20% sugar; about 2% to about 15% sugar; about 3% to about 10% sugar; about 4% to about 10% sugar; or about 5% to about 10% sugar. For example, the pharmaceutical formulations of the present invention may comprise about 0.5%; about 1.0%; about 1.5%; about 2.0%; about 2.5%; about 3.0%; about 3.5%; about 4.0%; about 4.5%; about 5.0%; about 5.5%; about 6.0%; 6.5%; about 7.0%; about 7.5%; about 8.0%; about 8.5%; about 9.0%; about 9.5%; about 10.0%; about 10.5%; about 11.0%; about 11.5%; about 12.0%; about 12.5%; about 13.0%; about 13,5%; about 14.0%; about 14.5%; about 15.0%; about 15.5%; about 16.0%; 16.5%; about 17.0%; about 17.5%; about 18.0%; about 18.5%; about 19.0%; about 19.5%; or about 20.0% sugar {e.g., sucrose).

The invention provides also the following non-limiting embodiments.

### EMBODIMENTS

1. A formulation which stabilizes an antibody composition, the formulation comprising (i) a pH buffered solution with a pH of about 5 to about 7.4, (ii) a polysorbate and (iii) one or more antibodies at a dose of less than about 70 μg, wherein the formulation is contained in a container means with a coating.
2. The formulation embodiment 1, wherein the buffer is histidine, phosphate, or acetate.
3. The formulation of embodiment 1, wherein the polysorbate is polysorbate 20 or polysorbate 80, and wherein the final concentration of the polysorbate in the formulation is at least 0.001% to 0.2% polysorbate weight/volume of the formulation.
4. The formulation of embodiment 1, wherein the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable injector pen.
5. The formulation of embodiment 1, wherein the container means comprises a silicon-dioxide coating or a hydrophobic coating.
6. The formulation of embodiment 1, wherein the pH buffered solution further comprises a sugar or a sugar alcohol.
7. The formulation of embodiment 1, wherein the pH buffered solution further comprises L-methionine or ethylenediaminetetraacetic acid
8. A formulation which stabilizes an anti-CD3 bispecific antibody composition, the formulation comprising (i) a pH buffered solution with a pH of about 5 to about 7.4 and (ii) a polysorbate, and (iii) an anti-CD3 bispecific antibody at a dose of less than about 70 μg wherein the formulation is contained in a container means with a coating.
9. The formulation of embodiment 8, wherein the buffer is histidine, phosphate, or acetate.
10. The formulation of embodiment 8, wherein the buffer is histidine, phosphate, or acetate.
11. The formulation of embodiment 8, wherein the polysorbate is polysorbate 20 or polysorbate 80, and wherein the final concentration of the polysorbate in the formulation is at least 0.001% to 0.2% polysorbate weight/volume of the formulation.
12. The formulation of embodiment 8, wherein the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable injector pen.
13. The formulation of embodiment 8, wherein the container means comprises a silicon-dioxide coating or a hydrophobic coating.
14. The formulation of embodiment 8, wherein the pH buffered solution further comprises a sugar or a sugar alcohol.
15. The formulation of embodiment 8, wherein the pH buffered solution further comprises L-methionine or ethylenediaminetetraacetic acid.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1: Adsorption Loss for an Antibody at Various Concentrations in Uncoated Glass Vials

Duvortuxizumab, a DART that targets CD19 and CD3, was filled into uncoated Type 1 glass 2R vials at different protein concentrations and fill volumes to evaluate the extent of adsorption loss at several dose levels: 3 μg, 7 μg, 30 μg, 70 μg, and 700 μg. The formulation contained 10 mM acetate, 0.01% (w/v) Polysorbate 80, 90 mg/mL sucrose, 0.4 mg/mL L-methionine, and 50 μM disodium EDTA at pH 5.2. The vials were placed on an orbital shaker set to 250 rpm for 3-4 days. The solutions were removed from the vials, and the protein concentration was compared to control samples based either on protein A HPLC measurements or intrinsic fluorescence. Table 1 indicates that duvortuxizumab recovery is much lower at low protein concentrations as compared to higher protein concentrations.

TABLE 1
Adsorption loss of duvortuxizumab for different
doses in uncoated glass vials.

| | | | Recovery in |
|---|---|---|---|
| | Initial protein | | uncoated |
| Dose per vial | concentration | Fill volume | Type 1 vial |
| (μg) | (mg/mL) | (mL) | (%) |
| 3 | 0.01 | 0.3 | 13 |
| 7 | 0.01 | 0.7 | 33 |
| 30 | 0.1 | 0.3 | 84 |
| 70 | 0.1 | 0.7 | 90 |
| 700 | 1 | 0.7 | 100 |

### Example 2: Influence of Nonionic Surfactants, Antibody, and Container Type on Adsorption

Ichorcumab, an anti-thrombin IgG4 antibody, was diluted to a protein concentration of 0.01 mg/mL in formulation buffers with or without 0.02% (w/v) Polysorbate 20 at three different pH values. The formulations contained 10 mM histidine and 8.5% (w/v) sucrose, at pH 5.0, 6.0, and 7.0. The diluted protein solutions were filled into 2R uncoated Type 1 glass vials (Schott Fiolax), 2R Schott Type 1 Plus coated vials, 2R Schott TopLyo coated vials or 0.5 mL Daikyo Crystal Zenith vials to a fill volume of 0.3 mL (30 μg Ichorcumab). Table 1 describes the materials for each of the vials. Each vial was placed on an orbital shaker set to 250 rpm for 3 days. The solutions were removed from the vials, and the protein concentration was compared to control samples based on intrinsic fluorescence.

Without Polysorbate 20 in the formulation, the protein adsorbed to all four vial types, regardless of pH (Table 3). With the addition of 0.02% (w/v) Polysorbate 20, the protein adsorbed to Type 1 glass in a pH-dependent manner, with more adsorption occurring at pH 5.0 and 6.0 and less adsorption occurring at pH 7.0. The isoelectric point of this protein is approximately 6.2. There was minimal adsorption loss of the protein in Type 1 Plus, TopLyo, or Crystal Zenith vials in the presence of 0.02% (w/v) Polysorbate 20.

TABLE 2
Characteristics of the four types of vials used.

| Vial material | Example |
|---|---|
| Uncoated Type 1 glass | Schott Fiolax ® |
| Type 1 glass with silicon-dioxide | Schott Type 1 Plus ® |
| coating | |
| Type 1 glass with hydrophobic | Schott TopLyo ® and |
| coating | TopYield ® |
| Cyclic polyolefin polymer | Daikyo Crystal |
| | Zenith ® |

TABLE 3
Adsorption loss of anti-thrombin IgG4 antibody in different
formulation buffers and in different containers.

| | Recovery in | | Recovery in | | Recovery in | | Recovery in | |
|---|---|---|---|---|---|---|---|---|
| | uncoated | | Type 1 Plus | | TopLyo | | Crystal | |
| | Type 1 vial | | coated vial | | coated vial | | Zenith vial | |
| Formulation | (%) | | (%) | | (%) | | (%) | |
| pH 5, no PS20 | 11 | (±1) | 12 | (±5) | 28 | (±7) | 81 | (±8) |
| pH 6, no PS20 | 15 | (±5) | 12 | (±2) | 17 | (±8) | 88 | (±4) |
| pH 7, no PS20 | 1 | (±2) | 9 | (±1) | 12 | (±13) | 89 | (±2) |
| pH 5, +0.02% PS20 | 2 | (±1) | 100 | (±5) | 100 | (±3) | 100 | (±4) |
| pH 6, +0.02% PS20 | 2 | (±3) | 99 | (±4) | 94 | (±1) | 95 | (±5) |
| pH 7, +0.02% PS20 | 11 | (±5) | 95 | (±2) | 96 | (±5) | 90 | (±6) |

In another experiment, duvortuxizumab, was evaluated at a concentration of 0.1 mg/mL in formulation buffers containing different levels of Polysorbate 80. The formulation contained 10 mM acetate, 9% sucrose, 0.4 mg/mL L-methionine, 50 μM disodium EDTA, and 0, 0.01% (w/v), and 0.1% (w/v) Polysorbate 80 at pH 5.2. The diluted protein solutions were filled into 2R uncoated Type 1 glass vials (Schott Fiolax), 2R Schott Type 1 Plus coated vials, or 2R Schott TopLyo coated vials to a fill volume of 0.3 mL (30 μg duvortuxizumab). The vials were placed on an orbital shaker set to 250 rpm for 3 days. The solutions were removed from the vials, and the protein concentration was compared to control samples based on protein A HPLC measurements.

Without Polysorbate 80 in the formulation, the protein adsorbed to all three vial types (Table 4). With the addition of 0.01% (w/v) Polysorbate 80, the protein adsorbed to Type 1 glass, but minimal adsorption loss occurred in the Type 1 Plus and TopLyo vials. The isoelectric point of the protein is approximately 8.7.

TABLE 4
Adsorption loss of duvortuxizumab in different formulation
buffers and in different containers.

| | Recovery in | Recovery in | Recovery in |
|---|---|---|---|
| | uncoated | Type 1 Plus | TopLyo |
| | Type 1 vial | coated vial | coated vial |
| Formulation | (%) | (%) | (%) |
| No PS80 | 36 | 61 | 76 |
| 0.01% PS80 | 84 | 99 | 97 |
| 0.1% PS80 | 87 | 100 | 100 |

In another experiment, several different antibodies were diluted to a protein concentration of 0.01 mg/mL in different formulations. Daratumumab, an anti-CD38 IgG1 antibody with an isoelectric point of approximately 8.4, was formulated with 10 mM histidine, 300 mM sorbitol, 0.04% (w/v) Polysorbate 20, 1 mg/mL L-methionine, at pH 5.6. Ustekinumab, an anti-IL12/IL23 IgG1 antibody with an isoelectric point of approximately 9, was formulated with 5.5 mM histidine, 0.004% (w/v) Polysorbate 80, 7.6% (w/v) sucrose, at pH 6. Sirukumab, an anti-IL6 IgG1 antibody with an isoelectric point of approximately 8, was formulated with 10 mM acetate, 5% (w/v) sorbitol, 0.04% (w/v) Polysorbate 20, at pH 5. A bispecific anti-IL1Rap x CD3 IgG4 antibody with an isoelectric point of approximately 7.8 was formulated with 10 mM acetate, 8% (w/v) sucrose, 0.04% (w/v) Polysorbate 20, 50 μM disodium EDTA, at pH 5.2. The diluted protein solutions were filled into 2R uncoated Type 1 glass vials (Schott Fiolax), 2R Schott Type 1 Plus coated vials, or 2R Schott TopLyo coated vials to a fill volume of 1.5 mL (15 μg total protein) and into 0.5 mL Daikyo Crystal Zenith vials to a fill volume of 0.3 mL (3 μg total protein). The vials were placed on an orbital shaker set to 250 rpm for 4 hours. The solutions were removed from the vials, and the protein concentration was compared to control samples based on intrinsic fluorescence.

In all four formulations, the pH was below the isoelectric point of the protein. Each of the four proteins adsorbed to Type 1 glass, but showed minimal adsorption loss in Type 1 Plus, TopLyo, or Crystal Zenith vials (Table 5).

TABLE 5
Adsorption loss of various antibodies
in four different vial containers.

| | Recovery in | | Recovery in | | Recovery in | | Recovery in | |
|---|---|---|---|---|---|---|---|---|
| | uncoated | | Type 1 Plus | | TopLyo | | Crystal | |
| | Type 1 vial | | coated vial | | coated vial | | Zenith | |
| Formulation | (%) | | (%) | | (%) | | (%) | |
| daratumumab | 68 | (±4) | 97 | (±3) | 100 | (±4) | 98 | (±7) |
| ustekinumab | 74 | (±7) | 95 | (±8) | 100 | (±9) | 100 | (±6) |
| sirukumab | 71 | (±4) | 100 | (±8) | 100 | (±2) | 100 | (±7) |
| Anti IL1RAP × | 57 | (±4) | 95 | (±7) | 100 | (±8) | 99 | (±8) |
| CD3 IgG4 | | | | | | | | |

### Example 3: Adsorbed Antibody as a Function of PH

An anti-NKG2D IgG4 antibody was diluted to a protein concentration of 0.01 mg/mL in three different formulation buffers (Table 6). Formulation buffer A contained 10 mM acetate, 0.04% (w/v) Polysorbate 20, 5% (w/v) sorbitol, at pH 5.0. Formulation buffer B contained 10 mM histidine, 0.04% (w/v) Polysorbate 20, 8.5% (w/v) sucrose, at pH 6.0. Formulation buffer C contained 10 mM phosphate, 0.04% (w/v) Polysorbate 20, 5% (w/v) sorbitol, at pH 7.4. The diluted protein solutions were filled into 2R uncoated Type 1 glass vials (Schott Fiolax), 2R Schott Type 1 Plus coated vials, or 2R Schott TopLyo coated vials to a fill volume of 1.5 mL (15 μg total anti-NKG2D) and into 0.5 mL Daikyo Crystal Zenith vials to a fill volume of 0.3 mL (3 μg total anti-NKG2D). The vials were placed on an orbital shaker set to 250 rpm for 4 hours. The solutions were removed from the vials, and the protein concentration was compared to control samples based on intrinsic fluorescence.

The isoelectric point of this protein is approximately 6.8, so buffers A and B are below the isoelectric point of the protein, while buffer C is above. The protein adsorbed to Type 1 glass in a pH-dependent manner, with more adsorption occurring at low pH and minimal adsorption occurring at pH 7.4. There was minimal adsorption loss of the protein in Type 1 Plus, TopLyo, or Crystal Zenith vials in all three formulations.

TABLE 6
Adsorption loss of anti-NKG2D IgG4 antibody in different
formulation buffers and in different containers.

| | Recovery in | | Recovery in | | Recovery in | | Recovery in | |
|---|---|---|---|---|---|---|---|---|
| | uncoated | | Type 1 Plus | | TopLyo | | Crystal | |
| | Type 1 vial | | coated vial | | coated vial | | Zenith | |
| Formulation | (%) | | (%) | | (%) | | (%) | |
| Buffer A at pH 5.0 | 73 | (±6) | 99 | (±4) | 100 | (±7) | 99 | (±11) |
| Buffer B at pH 6.0 | 91 | (±10) | 100 | (±4) | 100 | (±3) | 100 | (±7) |
| Buffer C at pH 7.4 | 100 | (±4) | 100 | (±5) | 99 | (±8) | 100 | (±11) |

### Example 4: Examination of Stability of Antibody Formulations Stored in Type 1 Plus Vials After 5 Months

Duvortuxizumab was diluted to a protein concentration of 0.1 mg/mL in formulation buffer containing 10 mM acetate, 0.01% Polysorbate 80, 9% sucrose, 0.4 mg/mL L-methionine, and 50 μM disodium EDTA at pH 5.2. The diluted protein solution was filled into 2R Schott Type 1 Plus vials to a fill volume of 0.3 mL (30 μg duvortuxizumab). The vials were stored at 5° C. for 5 months. The following attributes were tested: protein concentration by protein A HPLC, relative potency by antigen binding assays, percent monomer by SE-HPLC, purity by CE-SDS, and charge variants by IE-HPLC (Table 7).

TABLE 7
Stability of a bi-specific antibody at 0.1 mg/mL
stored in Type 1 Plus vials 5° C. for 5 months

| | | T = 5 |
|---|---|---|
| Test | T = 0 | months |
| Protein concentration (mg/mL) | 0.093 | 0.098 |
| Relative potency (%) | n/a | 100 |
| pH | 5.2 | 5.2 |
| SE-HPLC: % monomer | 99.9 | 99.7 |
| Reduced CE-SDS: % purity | 97.8 | 98.6 |
| Non-reduced CE-SDS: % purity | 96 | 97.8 |
| IE-HPLC: % Main peak | 52.4 | 54.8 |
| IE-HPLC: % Acidic variants | 27.5 | 25.1 |

### REFERENCES

WO2016048938
U.S. Pat. No. 9,518,129
U.S. Pat. No. 7,829,673
U.S. Pat. No. 6,902,734
U.S. Reissue 43,672
U.S. Pat. No. 7,879,985
WO2017079121
WO2017031104
WO2016036937
WO2016179518
WO2017127499
U.S. Pat. No. 5,153,072;
U.S. Pat. No. 5,002,794
US 2003/0092879
US 2002/0128234
U.S. Pat. No. 6,599,594
U.S. Pat. No. 6,844,028
U.S. Pat. No. 4,711,820
U.S. Pat. No. 8,025,915
U.S. Pat. No. 5,512,547
U.S. Pat. No. 5,756,468
U.S. Pat. No. 5,696,077
U.S. Pat. No. 6,235,340.
Chirakul P., Pérez-Luna V. H., Owen H., López G. P. Langmuir 2002, 18, 4324-4330.
Dissertation Thesis by Kierstin Hoger, Ludwig-Maximilians-Universität München, 2014.
Erika E. Johnston E. E., Bryers J. D., Ratner B. D. Langmuir 2005, 21, 870-881.
Hoger, Mathes & Friess, J Pharm Sci 104: 34-43, 2015.
Huang N. P., Michel R., Voros J., Textor M., Hofer R., Rossi A., Elbert D. L., Hubbell J. A., Spencer N. D. Langmuir 2001, 17, 489-498.
Jones & Kaufmann & Middaugh, J Pharm Sci 94: 918-927, 2005.
Kenausis G. L. Vörös J., Elbert D. L., Huang N., Hofer R., Ruiz-Taylor L., Textor M., Hubbell J. A., Spencer N. D. J. Phys. Chem. B 2000, 104, 3298-3309.
Lee S. W., Laibinis P. E. Biomaterials 1998, 19, 1660-1675Li L. Y., Chen S. F., Ratner B. D., Jiang S. Y. J. Phys. Chem. B 2005, 104, 2934-2941.
Lopez G. P., Ratner B. D. J. Polym. Sci. A—Polym. Chem. 1992, 30, 2415-2425.
Mathes & Friess, Eur J Pharm Biopharm 78: 239-47, 2011.
Pale-Grosdemange C., Simon F. S., Prime K. L., Whitesides G. M. J. Am. Chem. Soc. 1991, 113, 12-20.
Prime K. L., Whitesides G. M. J. Am. Chem. Soc. 1993, 115, 10714-10721.
Rabe, Verdes, & Seeger, Adv Colloid Interface Sci 162:87-106, 2011.
Sardella E., Gristina R., Senesi G. S., d'Agostino R., Favia P. Plasma Process. Polym. 2004, 1, 63-72.
Seigers C., Biesalski M., Haag R. Chem. Eur. J. 2004, 10, 2831-2838.
Shen M., Martinson L., Wagner M. S., Castner D. G., Ratner B. D., Horbett T. A. J. Biomater. Sci. Polymer Edn. 2002, 13, 367-390.
Shen M., Pan Y. V., Wagner M. S., Hauch K. D., Castner D. G., Ratner B. D., Horbett T. A. J. Biomater. Sci. Polymer Edn. 2001, 12, 961-978.
Sofia S. J., Premnath. V., Merrill E. W. Macromolecules 1998, 31, 5059-5070.
Yang Z., Galloway J. A., Yu H. Langmuir 1999, 15, 8405-8411.

We claim:

1. A formulation which stabilizes an antibody composition, the formulation comprising (i) a pH buffered solution with a pH of about 5 to about 7.4, (ii) a polysorbate and (iii) one or more antibodies at a dose of less than about 70 μg, wherein the formulation is contained in a container means with a coating.

2. The formulation claim 1, wherein the buffer is histidine, phosphate, or acetate.

3. The formulation of claim 1, wherein the polysorbate is polysorbate 20 or polysorbate 80, and wherein the final concentration of the polysorbate in the formulation is at least 0.001% to 0.2% polysorbate weight/volume of the formulation.

4. The formulation of claim 1, wherein the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable injector pen.

5. The formulation of claim 1, wherein the container means comprises a silicon-dioxide coating or a hydrophobic coating.

6. The formulation of claim 1, wherein the pH buffered solution further comprises a sugar or a sugar alcohol.

7. The formulation of claim 1, wherein the pH buffered solution further comprises L-methionine or ethylenediaminetetraacetic acid

8. A formulation which stabilizes an anti-CD3 bispecific antibody composition, the formulation comprising (i) a pH buffered solution with a pH of about 5 to about 7.4 and (ii) a polysorbate, and (iii) an anti-CD3 bispecific antibody at a dose of less than about 70 μg wherein the formulation is contained in a container means with a coating.

9. The formulation claim 8, wherein the buffer is histidine, phosphate, or acetate.

10. The formulation of claim 8, wherein the polysorbate is polysorbate 20 or polysorbate 80, and wherein the final concentration of the polysorbate in the formulation is at least 0.001% to 0.2% polysorbate weight/volume of the formulation.

11. The formulation of claim 8, wherein the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable injector pen.

12. The formulation of claim 8, wherein the container means comprises a silicon-dioxide coating or a hydrophobic coating.

13. The formulation of claim 8, wherein the pH buffered solution further comprises a sugar or a sugar alcohol.

14. The formulation of claim 8, wherein the pH buffered solution further comprises L-methionine or ethylenediaminetetraacetic acid.

15. A method for containing an antibody composition comprising providing an antibody composition ready for injection and comprising at least one antibody as an active ingredient at a dose of less than about 70 μg into a container means with a coating wherein the pharmaceutical composition is in a formulation comprising (i) a pH buffered solution with a pH of about 5 to about 7.4 and (ii) a polysorbate.
